(19) United States
(12) Patent Application Publication
(10) Pub. No.: US 2016/0303315 A1
(43) Pub. Date: Oct. 20, 2016

(54) INFUSION NEEDLE UNIT AND LIQUID INFUSION APPARATUS

(71) Applicant: Seiko Epson Corporation, Tokyo (JP)

(72) Inventor: Yoshihiko MOMOSE, Shiojiri (JP)

(21) Appl. No.: 15/095,433

(22) Filed: Apr. 11, 2016

(30) Foreign Application Priority Data
Apr. 14, 2015 (JP) 2015-082285

(51) Int. Cl. A61M 5/158; A61M 5/32; A61M 5/142

(52) U.S. Cl. CPC A61M 5/158; A61M 5/142; A61M 5/3298

(57) ABSTRACT

A liquid infusion apparatus includes an infusion needle in which a flow path of a liquid is provided, a supporting member having elasticity and supporting a position between a tip end and a base end of the infusion needle in a longitudinal direction, a regulating member that regulates a movement of the base end of the infusion needle, and a pump that transports the liquid.

### BACKGROUND

1. Technical Field

The present invention relates to an infusion needle unit and a liquid infusion apparatus.

2. Related Art

Recently, as a method of treatment for diabetes, continuous subcutaneous insulin infusion therapy (CSII therapy) has attracted attention. The continuous subcutaneous insulin infusion therapy is a method of treatment of inserting and indwelling an infusion needle (cannula) under the skin by a patient himself or herself and continuously infusing insulin from a portable liquid reservoir via the infusion needle. In the background, various easy-to-use liquid infusion apparatuses are studied (for example, see Patent Document 1 (JP-A-2014-42669)).

The liquid infusion apparatus is indwelled in the skin (hereinafter, referred to as “living body”) during activity of the living body for continuous supply of a chemical and has various requests different from those for common syringes. Particularly, it is known that the skin routinely expands and contracts according to the joint movement, the breathing movement, the dry condition of the stratum corneum of the epidermis, etc. Accordingly, if the infusion needle indwelled in the skin does not move with the expansion and contraction of the skin, the infusion needle causes excessive stress on the punctured region of the skin (hereinafter, referred to as “punctured region”) and moves its needle tip within the punctured region, and thereby, may cause pain and discomfort to the patient. This problem is particularly readily caused in the case where a plurality of needles are provided. If the needles are fixed with respect to each other, when one infusion needle is to move with the movement of the skin in the punctured region, the other infusion needle restricts the movement, and excessive stress may act on the punctured region.

Patent Document 1 describes a configuration in which the infusion needle is made shorter in order to reduce the pain at puncture etc., however, does not describe the pain and discomfort due to expansion and contraction of the skin when the infusion needle is indwelled.

### SUMMARY

An advantage of some aspects of the invention is to provide an infusion needle unit and a liquid infusion apparatus that are harder to cause discomfort and pain to a patient when an infusion needle is indwelled.

An aspect of the invention is directed to an infusion needle unit including an infusion needle in which a flow path of a liquid is provided, a supporting member having elasticity and supporting a position between a tip end and a base end of the infusion needle in a longitudinal direction, and a regulating member that regulates a movement of the base end of the infusion needle. The other features of the invention will be disclosed by the description of the accompanying drawings and the specification.

### BRIEF DESCRIPTION OF THE DRAWINGS

The invention will be described with reference to the accompanying drawings, wherein like numbers reference like elements.

FIGS. 1A and 1B show an appearance of a liquid infusion apparatus in a first embodiment of the invention.

FIG. 2 is a sectional view showing an internal configuration of the liquid infusion apparatus in the first embodiment of the invention.

FIG. 3 is a sectional view showing the internal configuration of the liquid infusion apparatus in the first embodiment of the invention.

FIG. 4 shows a configuration of an infusion needle unit in the first embodiment of the invention.

FIG. 5 shows a configuration of the infusion needle unit in the first embodiment of the invention.

FIG. 6 shows an indwelling state of an infusion needle in the first embodiment of the invention.

FIG. 7 shows a movement mode of the infusion needle in the first embodiment of the invention.

FIG. 8 shows a movement mode of the infusion needle in the first embodiment of the invention.

FIG. 9 shows a movement mode of the infusion needle in the first embodiment of the invention.

FIG. 10 shows a movement mode of the infusion needle when a skin expands and contracts in the first embodiment of the invention.

FIG. 11 shows a movement state of the infusion needle at puncture in the first embodiment of the invention.

FIG. 12 shows a configuration of an infusion needle unit in a second embodiment of the invention.

FIG. 13 shows a configuration of an infusion needle unit in another embodiment of the invention.

### DESCRIPTION OF EXEMPLARY EMBODIMENTS

At least the following matters will be clear from the description of the specification and the accompanying drawings.

An infusion needle unit including an infusion needle in which a flow path of a liquid is provided, a supporting member having elasticity and supporting a position between a tip end and a base end of the infusion needle in a longitudinal direction, and a regulating member that regulates a movement of the base end of the infusion needle is disclosed. According to the infusion needle unit, the infusion needle unit that is harder to cause discomfort and pain to a patient when the infusion needle is indwelled may be obtained. Further, a range in which the infusion needle moves may be regulated by the regulating member, and thereby, slipping out during indwelling may be prevented.

It is desirable that the liquid holding part that holds the liquid while supporting the supporting member and the regulating member is provided, wherein the infusion needle is provided with an inflow port communicating with the flow path in the base end, and the liquid held in the liquid holding part flows into the infusion needle from a gap between the base end of the infusion needle and the regulating member via the inflow port. According to the infusion needle unit, stress in a vertical direction on the infusion needle caused at puncture may be relaxed.

It is desirable that the regulating member regulates a movement of the infusion needle from the tip end toward the base end. According to the infusion needle unit, an amount of movement of the infusion needle in the vertical direction is regulated at puncture, and thereby, the puncture may be reliably made and slipping out may be prevented.

It is desirable that the supporting member supports the infusion needle so that the base end of the infusion needle may move in contact with the regulating member, the base end of the infusion needle takes on a curved shape convex outward, and a surface of the regulating member in contact with the base end of the infusion needle takes on a curved shape concave inward. According to the infusion needle unit, stability of the position of the infusion needle may be improved.

It is desirable that the infusion needle is provided with an inflow port that allows the liquid to flow into the flow path between the position supported by the supporting member and the base end. According to the infusion needle unit, the inflow port may be prevented from being blocked by the regulating member and the liquid may be reliably supplied.

It is desirable that the supporting member supports a plurality of the infusion needles. According to the infusion needle unit, the amount of supply per unit time may be increased while the outer shape of the infusion needle is reduced. In this case, stress on a punctured region from the tip end of the infusion needle generated due to expansion and contraction of a skin may be relaxed.

A liquid infusion apparatus including an infusion needle in which a flow path of a liquid is provided, a supporting member having elasticity and supporting a position between a tip end and a base end of the infusion needle in a longitudinal direction, a regulating member that regulates a movement of the base end of the infusion needle, and a pump that transports the liquid is disclosed. According to the liquid infusion apparatus, the liquid infusion apparatus that is harder to cause discomfort and pain to a patient when the infusion needle is indwelled may be obtained. Further, a range in which the infusion needle moves may be regulated by the regulating member, and thereby, slipping out during indwelling may be prevented.

### First Embodiment

In the embodiment, a liquid infusion apparatus used for insulin infusion therapy is explained as an example.

### Basic Configuration of Liquid Infusion Apparatus

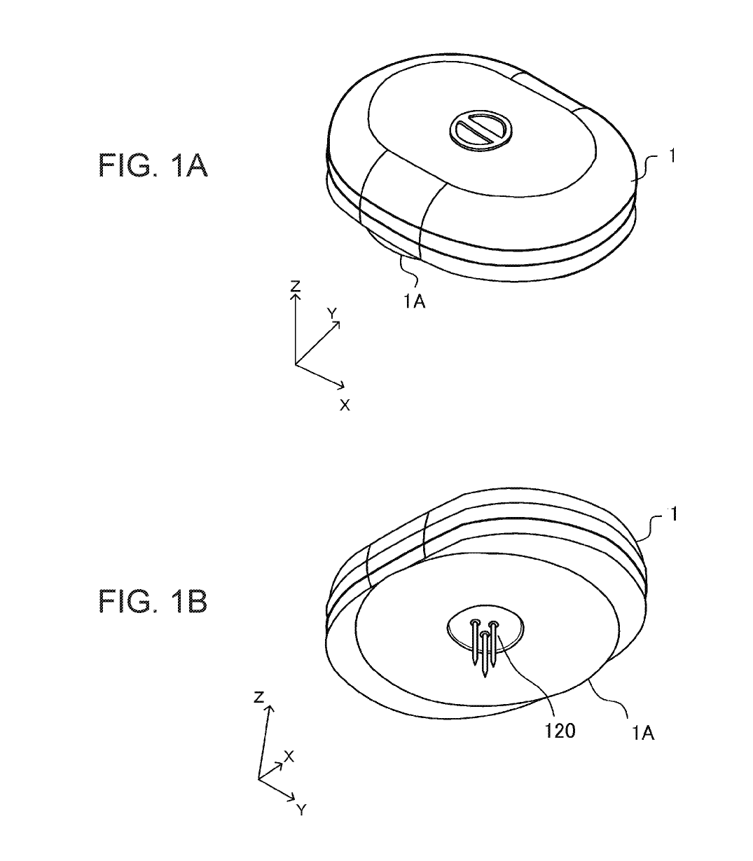

FIGS. 1A, 1B, 2, 3 show an example of a configuration of the liquid infusion apparatus according to the embodiment.

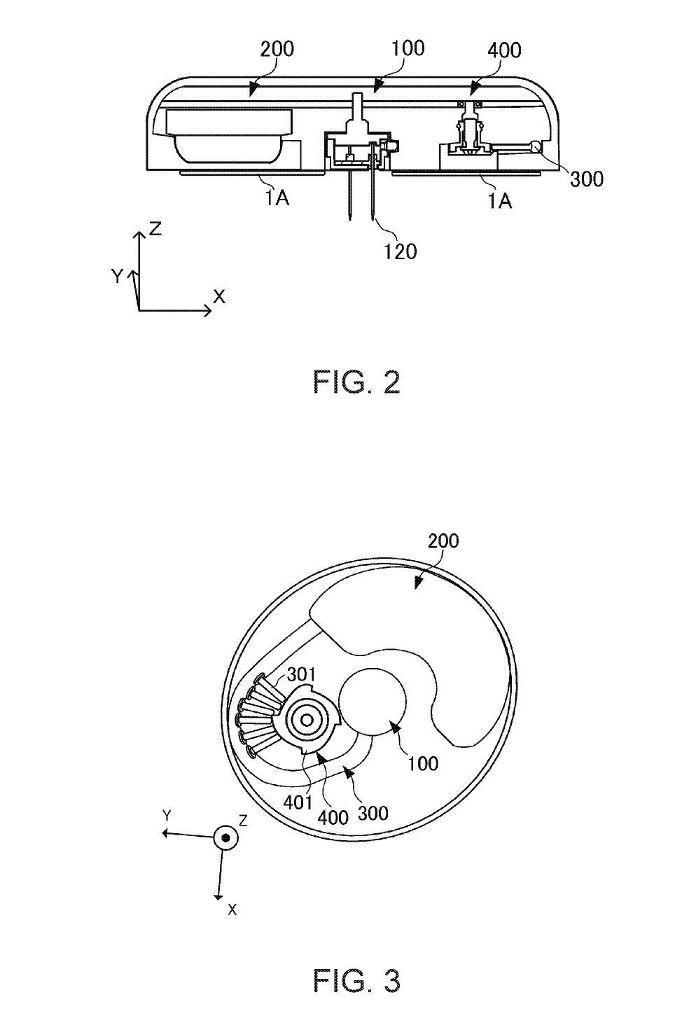

FIG. 1A is a perspective view showing an appearance when the liquid infusion apparatus is seen from above, and FIG. 1B is a perspective view showing the appearance when the liquid infusion apparatus is seen from below. FIG. 2 is a sectional view of an internal configuration of the liquid infusion apparatus as seen from a side, and FIG. 3 is a sectional view of the internal configuration of the liquid infusion apparatus as seen from above. Note that, in the drawings, an X-axis, a Y-axis, a Z-axis show directions for clarification of position relationships among respective members in the respective drawings. With a direction pointed by the Z-axis (an opposite direction to a direction in which a tip of an infusion needle 120 extends) as an upward direction, the X-axis shows a direction from a position of a liquid reservoir part 200 to a position of an infusion needle unit 100, and the Y-axis shows a direction orthogonal to the X-axis and the Z-axis (the same applies to the following description).

The liquid infusion apparatus according to the embodiment is an apparatus that retains insulin (hereinafter, referred to as “liquid”) within a casing 1 and infuses the liquid into a living body using the infusion needle 120 exposed under the casing 1.

The liquid infusion apparatus has a configuration in which an adhesive pad (fixing member) 1A is attached to the lower part of the casing 1 and the tip of the infusion needle 120 protrudes downward from the adhesive pad 1A (see FIGS. 1A, 1B). The adhesive pad 1A has an adhesive surface on the lower side and the adhesive surface is attached to a region to which the liquid is infused (e.g. an abdomen of a human body), and thereby, the liquid infusion apparatus is fixed to the living body. That is, the liquid infusion apparatus is fixed to the living body by the adhesive pad 1A, and thereby, even during activity of the living body, the infusion needle 120 is held in a position in which the needle punctures the region to which the liquid is infused. Note that, when the infusion needle 120 is inserted, for example, a patient himself or herself presses the tip of the infusion needle 120 against the region to be infused.

The liquid infusion apparatus includes the infusion needle unit 100, the liquid reservoir part 200, a supply tube 300, and a liquid transport part 400 within the casing 1.

The infusion needle unit 100 is a unit that holds the infusion needle 120 and supplies the liquid to the living body via the infusion needle 120. The infusion needle unit 100 is provided on the center bottom surface within the casing 1 and holds the infusion needle 120 so that its tip may protrude from the lower part of the casing 1. The infusion needle unit 100 receives the liquid from the liquid reservoir part 200 via the supply tube 300, and transports the liquid to the living body in a hollow region inside of the infusion needle 120 as a flow path. The detailed explanation of the infusion needle unit 100 will be made later.

The liquid reservoir part 200 is a container that retains the liquid and a unit that supplies the liquid to the infusion needle unit 100 via the supply tube 300.

The supply tube 300 is a flow path for introducing the liquid from the liquid reservoir part 200 to the infusion needle unit 100 and includes e.g. a rubber tube. The supply tube 300 has one end connected to the supply port of the liquid reservoir part 200 and the other end connected to a receiving port 110A of the infusion needle unit 100, and moves the liquid from the side of the liquid reservoir part 200 to the side of the infusion needle unit 100. Further, the supply tube 300 is provided within the casing 1 along the outer circumference of a disc-shaped cam 401 to be pressed when the disc-shaped cam 401 rotates.

The liquid transport part 400 is a pump unit that transports the liquid retained in the liquid reservoir part 200 to the infusion needle unit 100 and includes e.g. a cam mechanism. The cam mechanism includes the disc-shaped cam 401 partially projecting in the outer circumferential direction. Further, the liquid transport part 400 rotates the disc-shaped cam 401 in a direction from the upstream side (the liquid reservoir part 200) toward the downstream side (the infusion needle unit 100) of the supply tube 300, and thereby, the projecting portion of the disc-shaped cam 401 compresses the supply tube 300 from the upstream side toward the downstream side and moves the liquid within the supply tube 300 to express the liquid from the upstream side toward the downstream side. The liquid transport part 400 adjusts the rotation speed of the disc-shaped cam 401, and thereby, adjusts the amount of liquid transported to the infusion needle unit 100. Note that a plurality of fingers 301 in contact with the disc-shaped cam 401 are attached to the supply tube 300, and the supply tube 300 is pressed against the inner wall side of the casing 1 via the fingers 301 and compressed so that the diameter may be smaller in response to the rotation of the disc-shaped cam 401.

### Detailed Configuration of Infusion Needle Unit

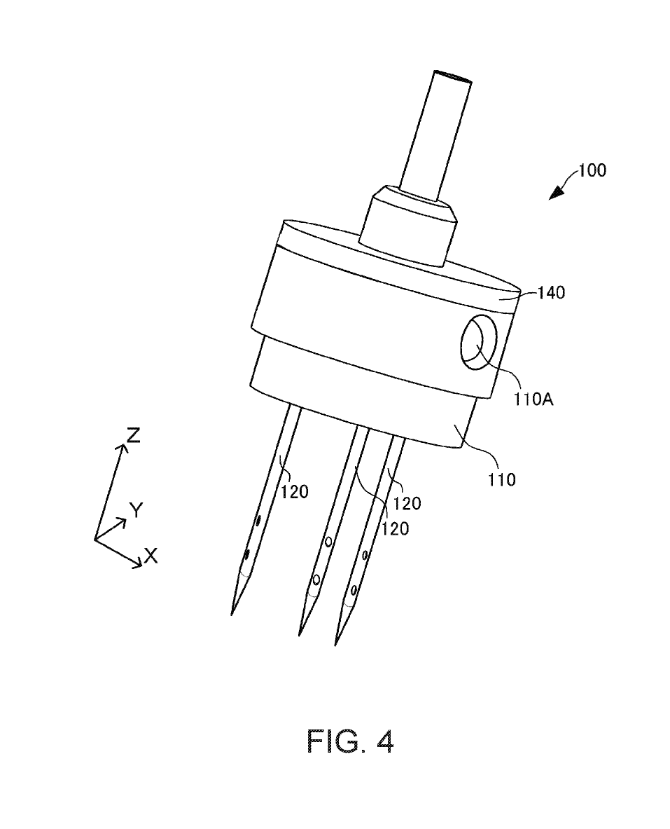

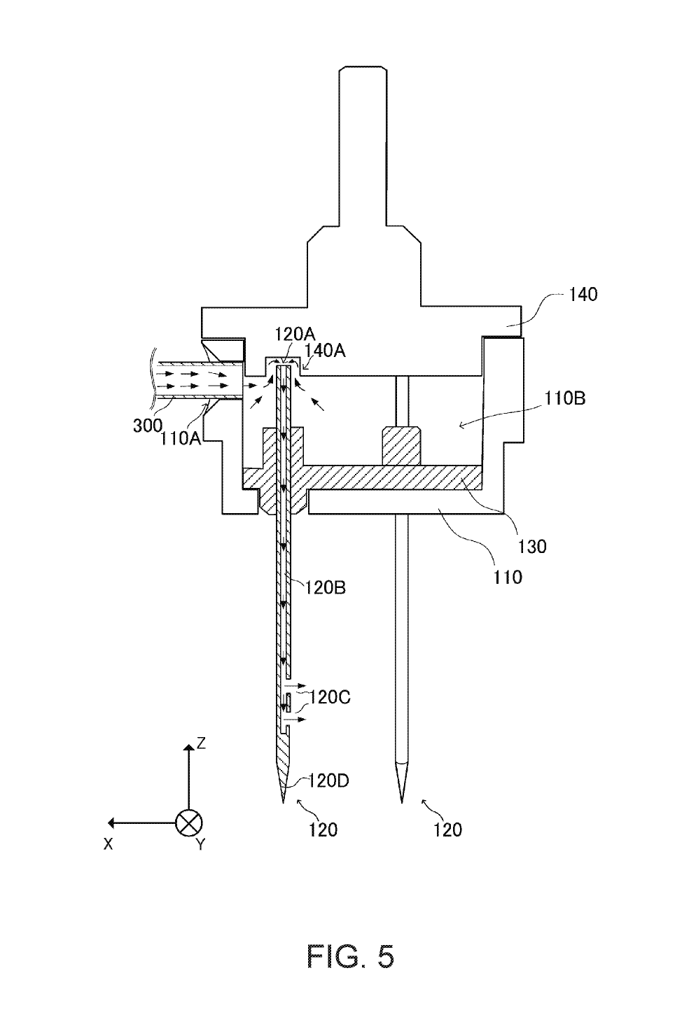

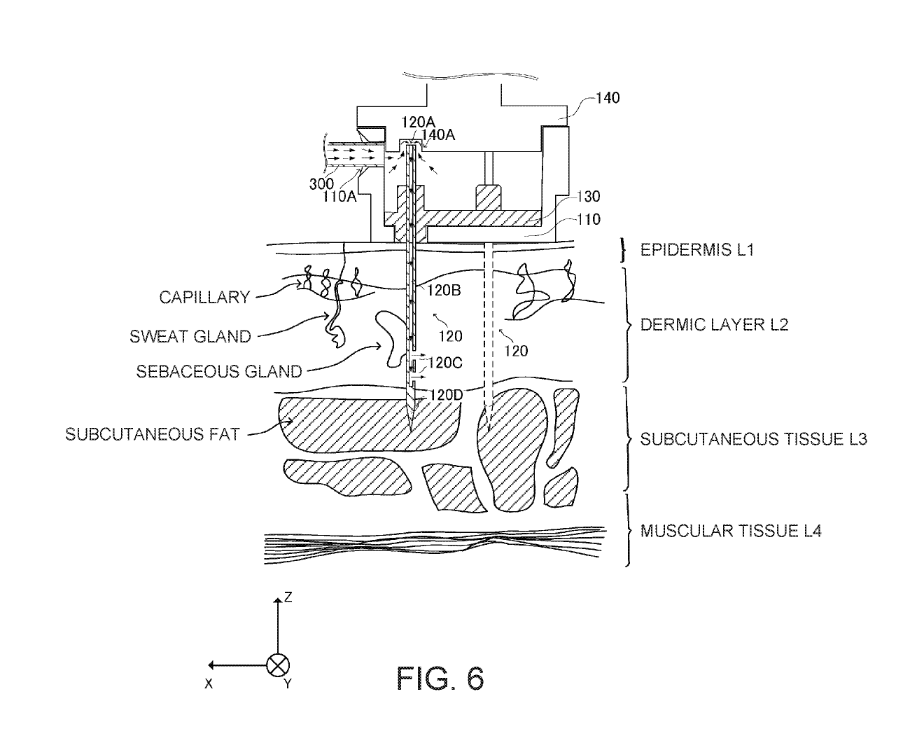

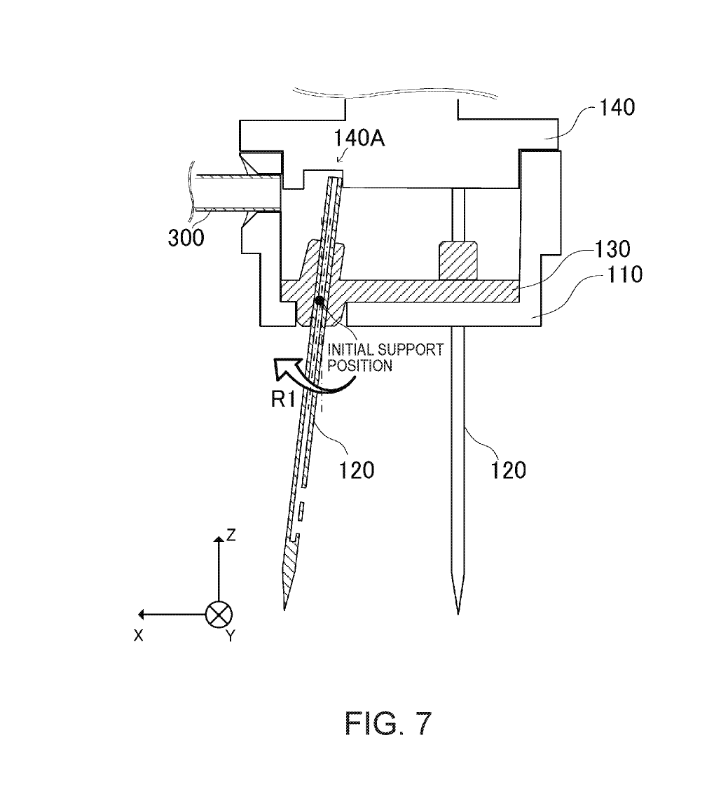

Next, referring to FIGS. 4 to 9, an example of a configuration of the infusion needle unit 100 according to the embodiment is shown. FIG. 4 is a perspective view of an appearance of the infusion needle unit 100 as seen from above. FIG. 5 is a sectional view of the infusion needle unit 100 as seen from a side. FIG. 6 shows a state in which the infusion needle 120 of the infusion needle unit 100 is indwelled in the living body. FIGS. 7 to 9 are diagrams for explanation of movement modes of the infusion needle 120. Note that FIGS. 5 and 6 show a state in which the supply tube 300 is inserted into the receiving port 110A of the infusion needle unit 100. Further, arrows in FIGS. 5 and 6 show flow paths of the liquid.

The infusion needle unit 100 includes a liquid holding part 110, the infusion needle 120, a supporting member 130, and a regulating member 140, and is formed by attachment of the infusion needle 120 and the supporting member 130 to the lower part of the liquid holding part 110 and attachment of the regulating member 140 to the upper part of the liquid holding part 110. The infusion needle unit 100 receives the liquid from the supply tube 300 via the receiving port 110A formed in the liquid holding part 110, and infuses the liquid into the living body from an outflow port 120C formed in the infusion needle 120. Note that the regulating member 140 according to the embodiment has a role of regulating the movement of the infusion needle 120 within a fixed range and a role of air-tightly closing a housing space 110B by covering the upper part of the liquid holding part 110.

Liquid Holding Part

The liquid holding part 110 is a container that receives the liquid from the supply tube 300 and temporarily holds the liquid. The liquid holding part 110 supports the regulating member 140 in the upper part and a plurality of the infusion needles 120 and the supporting member 130 in the lower part, and forms the housing space 110B air-tightly closed by these members (see FIG. 5). Further, the liquid holding part 110 has the receiving port 110A on the side surface and the support port of the supply tube 300 is inserted into the receiving port 110A. The liquid holding part 110 receives the liquid from the supply tube 300 and holds the liquid in an amount that may fill the housing space 110B. When the liquid is supplied to the amount that may fill the housing space 110B, the liquid filling the housing space 110B moves from an inflow port 120A formed on the rear end side of the infusion needle 120 to a needle part flow path 120B inside of the infusion needle 120. Note that a stopper in contact with the supply tube 300 at insertion of the tube is formed (the side surface part of the regulating member 140) in the receiving port 110A of the liquid holding part 110, and the supply tube 300 is inserted into the receiving port 110A to press the stopper into close contact.

### Infusion Needle

The infusion needle 120 is a needle indwelled in the punctured region of the living body and infusing the liquid into the living body via the needle part flow path 120B inside. The infusion needle 120 is supported by the supporting member 130 in the position between the tip end and the base end in the longitudinal direction so that the tip end may project from the bottom surface of the casing 1 (the liquid holding part 110). The base end of the infusion needle 120 is provided in a position surrounded by a wall surface of a concave groove portion 140A within the housing space 110B of the liquid holding part 110 (the part on the punctured side of the infusion needle 120 in the longitudinal direction is referred to as “tip end” and the opposite part is referred to as “base end”, and the same applies to the following description). The infusion needle 120 is movably supported by the supporting member 130 (details of actions of the infusion needle 120 will be described later).

The infusion needle 120 includes the inflow port 120A on the base end side, the needle part flow path 120B inside, and the outflow port 120C on the tip end side, and forms the liquid flow path by the inflow port 120A, the needle part flow path 120B, and the outflow port 120C (see FIG. 5). The needle part flow path 120B is a hollow region formed inside of the member forming the infusion needle 120 to extend from the base end side and the tip end side. Further, the needle part flow path 120B is formed to allow the inflow port 120A and the outflow port 120C to communicate with each other and forms a liquid flow path inside of the infusion needle 120. Note that the inflow port 120A and the outflow port 120C are openings formed on the base end side to the tip end side of the infusion needle 120, respectively, and flow paths formed to communicate with the hollow space of the needle part flow path 120B.

The infusion needle 120 includes a non-hollow puncture portion 120D at the tip end side with respect to the needle part flow path 120B and the outflow port 120C. With the outflow port 120C as a configuration penetrating the side wall of the member in a lateral direction (a direction orthogonal to the longitudinal direction of the infusion needle 120), the infusion needle 120 supplies a liquid to a position near the surface of the living body while indwelling the puncture portion 120D in a deeper position of the surface of the living body, and thereby, improves the safety of the indwelling state by the configuration. The dimensions of the infusion needle 120 are, in the case of insulin infusion to a dermic layer, which will be described later, e.g. 5.0 mm or less in the whole length including the puncture portion 120D, 0.18 mm in the outer diameter, and 0.11 mm in the inner diameter of the hollow region.

The infusion needle 120 is formed using e.g. titanium as a material having good biological compatibility. As the material forming the infusion needle 120, any other material than titanium may be used as long as the material has rigidity at a degree that can penetrate the epidermis. For example, a metal material such as stainless or a resin material such as Teflon (registered trademark) may be used. For protection of biological tissues, the other region of the infusion needle 120 than the outflow port 120C may be covered by a soft resin material. As a method of forming the infusion needle 120, any method that can mold a minute shape including integral processing such as metal powder injection molding or resin molding and a combination of tip processing (e.g. swaging processing on a tip of a pipe) and lateral hole processing (laser processing, punch processing, discharge processing) may be used.

The infusion needle unit 100 includes a plurality of (here, three) infusion needles 120, and infuses a liquid into the living body by puncturing the living body with the plurality of infusion needles 120. The infusion needles 120 are inserted into the living body and indwelled there, and their outer shapes are required to be as thin as possible. On the other hand, particularly, in the insulin infusion therapy, it is necessary to infuse insulin in amounts according to blood sugar levels changing over time, and it is desirable to expand the adjustment range of the amount of supply of insulin. Accordingly, in the infusion needle unit 100 according to the embodiment, the plurality of infusion needles 120 are provided, and thereby, the amount of supply of insulin per unit time is increased while the outer shapes of the infusion needles 120 are made thinner.

### Supporting Member

The supporting member 130 is a member that movably supports the infusion needle 120. The supporting member 130 is attached to the lower part of the liquid holding part 110 and supports the infusion needle 120 in a position between the tip end and the base end of the infusion needle 120. Further, the supporting member 130 supports the infusion needle 120 so that the tip end of the infusion needle 120 may protrude from the bottom surface of the casing 1 and the base end of the infusion needle 120 may be surrounded by the wall surface of the concave groove portion 140A in the housing space 110B. Further, the supporting member 130 supports the infusion needle 120 to provide a gap between the base end of the infusion needle 120 and the wall surface of the groove portion 140A so that the infusion needle 120 may move in the vertical direction (upwardly).

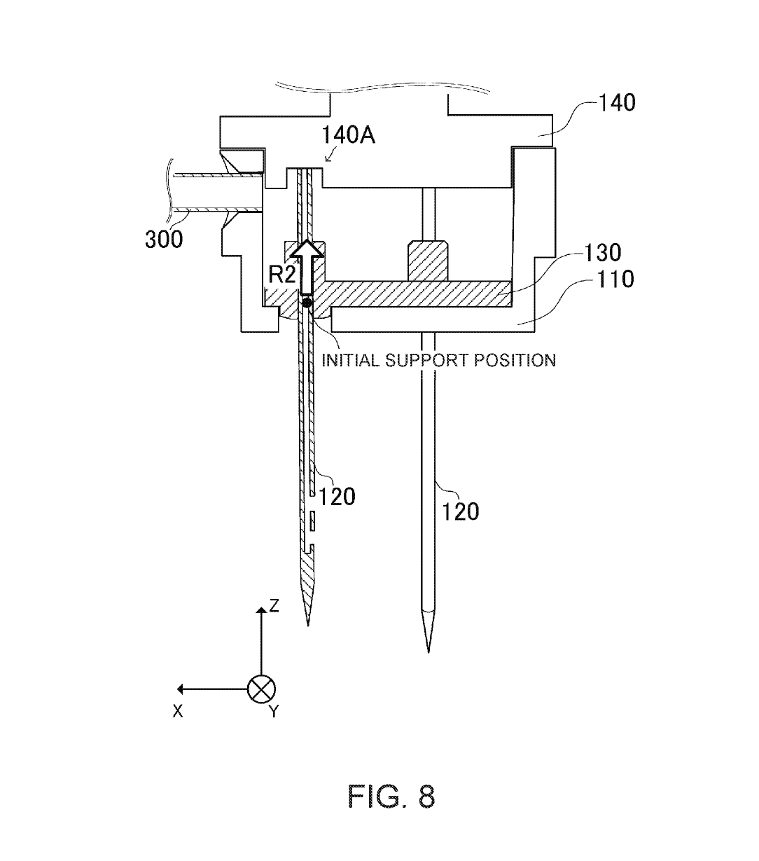

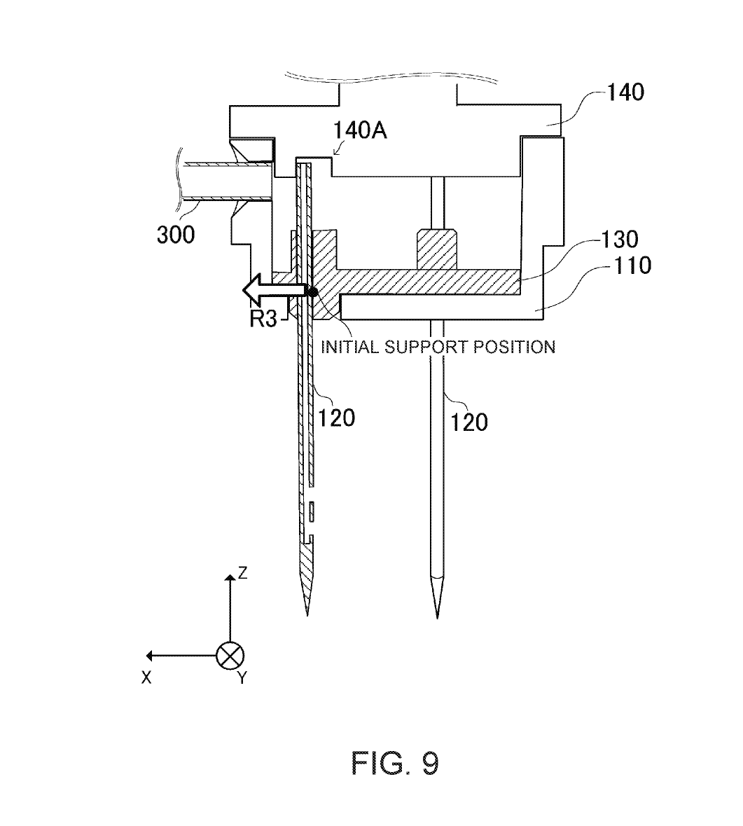

The supporting member 130 includes a member having elasticity of rubber, elastomer, or the like and elastically deforms according to stress acting from the infusion needle 120. In other words, the infusion needle 120 is supported to elastically deform the supporting member 130 according to stress from the punctured region so as to move within a fixed range from the position where the needle is supported without stress (hereinafter, referred to as “initial support position”). The infusion needle 120 is supported in the position between the tip end and the base end surrounded by the elastically deforming supporting member 130, and thereby, movable in rotation directions, horizontal directions, and vertical directions from the initial support position. The infusion needle 120 is inserted to penetrate the elastic member of e.g. rubber or elastomer from above toward below and the elastic member and the infusion needle 120 are bonded, and thereby, the support ing member 130 supports the infusion needle 120 to surround the position between the tip end and the base end. Note that “the infusion needle 120 is movable” means that the position of the infusion needle 120 may change relatively to the initial support position of the supporting member 130. Here, the rotation directions refer to directions in which the infusion needle 120 tilts about the initial support position as a fulcrum (see FIG. 7), the vertical directions refer to longitudinal directions of the infusion needle 120 with respect to the initial support position (see FIG. 8). Further, the horizontal directions refer to directions parallel to the surface of the outermost layer of the skin of the punctured region and, in the embodiment, refer to vertical directions to the longitudinal directions of the infusion needle 120 with respect to the initial support position (see FIG. 9).

The supporting member 130 supports the plurality of infusion needles 120. The supporting member 130 supports the plurality of infusion needles 120 so that the respective tip ends of the plurality of infusion needles 120 may protrude below and the base ends may be surrounded by the wall surfaces of the concave groove portions 140A. Further, the plurality of infusion needles 120 are respectively supported in the positions between the tip ends and the base ends surrounded by the elastically deforming supporting member 130, and thereby, independently movable in rotation directions, horizontal directions, and vertical directions from the initial support position.

### Regulating Member

The regulating member 140 is a member that regulates the movement range of the infusion needle 120. The regulating member 140 is provided on the base end side of the infusion needle 120 and, when the infusion needle 120 moves in a certain amount, comes into contact with the base end of the infusion needle 120, and thereby, regulates the movement range of the infusion needle 120 as a stopper. That is, the infusion needle 120 is movably supported by the supporting member 130, and may slip out if there is no constraint of the movement range. Accordingly, the regulating member 140 regulates the movement of the base end of the infusion needle 120, and thereby, regulates the movement range of the infusion needle 120 (the details will be described later).

Specifically, the regulating member 140 is formed to cover the upper part of the housing space 110B of the liquid holding part 110. The regulating member 140 forms the concave groove portion 140A in the position opposed to the base end of the infusion needle 120 on the lower surface side and surrounds the base end of the infusion needle 120 by the wall surface of the groove portion 140A, and thereby, regulates the movement range of the base end of the infusion needle 120. That is, the regulating member 140 surrounds the base end of the infusion needle 120 by the wall surface of the groove portion 140A, and thereby, when the base end of the infusion needle 120 moves in a certain amount in one direction of the horizontal directions, vertical directions, and rotation directions, regulates the movement by bringing the base end of the infusion needle 120 into contact with the wall surface of the groove portion 140A. For example, when the infusion needle 120 moves (rotates) in a certain amount to float the tip end, the base end comes into contact with the wall surface of the groove portion 140A to be not movable (rotatable) to float the tip end more than that, and thereby, the movement range is regulated. The regulating member 140 forms the groove portions 140A in positions opposed to the respective base ends of the plurality of infusion needles 120 (not shown).

Note that the concave groove portion 140A has a groove shape larger than the shape of the base end of the infusion needle 120, and the groove shape is set in consideration of the range in which the infusion needle 120 is movable (the regulation range) so that, when the base end of the infusion needle 120 moves, the base end may come into contact with the wall surface of the groove portion 140A and the movement of the base end of the infusion needle 120 may be regulated. Here, “regulation” means that the base end of the infusion needle 120 is movable only in a predetermined range, but not movable beyond the predetermined range.

The regulating member 140 is desirably formed using a material having higher rigidity than the elastic member forming the supporting member 130, e.g. a rubber member having higher rigidity (urethane rubber) so as to regulate the movement of the base end of the infusion needle 120. Further, in the embodiment, the regulating member 140 also has a role of a lid member that covers the upper part of the housing space 110B of the liquid holding part 110, and thus, the member is attached to the upper part of the liquid holding part 110 in close contact by e.g. welding for sealing so that the liquid may not leak from the housing space 110B to the other region.

### Action of Infusion Needle Unit

Next, referring to FIGS. 6, 10, 11, the action of the infusion needle unit 100 will be explained.

### Flow Path of Liquid

A skin includes an epidermis L1, a dermic layer L2, a subcutaneous tissue L3, and a muscle tissue L4 sequentially from the outermost layer (see FIG. 6). As a technique of transdermal delivery of a chemical using a syringe needle, generally, a method of administrating the chemical to the subcutaneous tissue L3 and the muscle tissue L4 into which the syringe needle can be inserted is used. However, it has been clear that the dermic layer L2 existing beneath the epidermis L1 is a part having many capillaries and effective for absorption of insulin, glucagon, growth hormone etc. In addition, when insulin is continuously infused into the subcutaneous fat of the subcutaneous tissue L3, insulin balls by hyperplasia and induration of subcutaneous fat are produced and the absorption efficiency of insulin becomes lower. On the other hand, the dermic layer L2 existing beneath the epidermis L1 is in a shallow region from the surface of the skin at a depth from about 0.5 mm to 2.5 mm from the outermost layer of the epidermis L1 (there are some differences depending on physical build and race), and thus, when the needle is inserted only to the depth, the needle easily slips out and, when the living body is active, the needle is harder to indwell.

Accordingly, the infusion needle unit 100 according to the embodiment has a more preferable structure for continuous supply of the liquid to the position closer to the living body, particularly, to the dermic layer L2 by the above described configuration.

As described above, the liquid is supplied from the liquid reservoir part 200 via the supply tube 300 to the liquid holding part 110 of the infusion needle unit 100 by the operation of the liquid transport part 400. Then, the liquid supplied to the liquid holding part 110 is accumulated to in the amount that may fill the housing space 110B of the infusion needle unit 100 and flows into the inflow port 120A of the infusion needle 120 from the gap between the base end of the infusion needle 120 and the wall surface of the groove portion 140A. Then, the liquid flowing into the inflow port 120A of the infusion needle 120 moves to the tip end side via the needle part flow path 120B and is supplied to the living body via the outflow port 120C.

Here, in the infusion needle 120, the outflow port 120C is formed in the lateral direction (nearly perpendicular to the longitudinal direction) with respect to the needle part flow path 120B, and thereby, the liquid may be supplied to the position closer to the surface of the living body (dermic layer L2). Further, a plurality of the infusion needles 120 are provided in the infusion needle unit 100 and a plurality of outflow ports 120C are formed in each of the plurality of infusion needles 120, and thereby, the liquid is supplied from the respective outflow ports 120C to spread into the dermic layer L2 and the liquid is efficiently absorbed in the dermic layer L2. Furthermore, the infusion needle 120 supplies the liquid only from the outflow ports 120C, but does not supply the liquid from the tip end. Accordingly, the liquid is supplied only to the dermic layer L2 with higher absorption efficiency and the supplied amount of the liquid is fully absorbed by the living body. In other words, the amount of infusion into the living body may be appropriately adjusted by the configuration.

The positions in which the outflow ports 120C are provided are set such that the dimensions from the part defining the position of the outermost layer of the epidermis of the living body (e.g. the adhesive surface of the adhesive pad 1A) to the outflow ports 120C may be from 0.5 mm to 2.5 mm (corresponding to the depth of the dermic layer L2 of the living body) so that, when the infusion needle 120 is inserted, the liquid is infused into the dermic layer L2. Note that formation of the outflow ports 120C in the lateral direction refers to formation of the ports from the needle part flow path 120B to penetrate the side wall of the member of the infusion needle 120, and the ports may be formed in an oblique direction as long as the direction is nearly perpendicular direction to the longitudinal direction.

Further, the infusion needle 120 is extended to the subcutaneous tissue L3 deeper than the dermic layer L2 by the puncture portion 120D nearer the tip end side than the outflow port 120C, and thereby, the infusion needle 120 is stably held not to slip out from the dermic layer L2. Particularly, the puncture portion 120D is provided, and thereby, the puncture to the depth nearly twice the depth of the dermic layer L2 can be made and stability of the indwelling state may be improved against the friction force acting between the cloths and the casing 1 of the liquid infusion apparatus (the force acting in the direction perpendicular to the longitudinal direction of the infusion needle 120) when the living body is active. In addition, the puncture portion 120D has no opening in the tip end unlike the normal syringe needle, and thus, when the needle is inserted, the biological tissue compressed and affected by the tip end portion of the needle (specifically, the edge portion of the opening) may be suppressed. Further, the puncture portion 120D of the infusion needle 120 is formed in a circular conical shape, and thereby, the puncture resistance at puncture into the living body may be reduced and the pain at puncture may be reduced.

That is, if a needle with a hole opening on the tip is not inserted into the subcutaneous tissue L3, but inserted into the epidermis L1 and the dermic layer L2, the needle is short and indwelling of the needle is unstable. Or, if a needle with a hole on the tip is inserted into the epidermis L1, the dermic layer L2, and the subcutaneous tissue L3, indwelling of the needle is stable, but control of the amount of infusion of insulin into the dermic layer L2 is harder because insulin flows out from the hole on the tip. On the other hand, in the embodiment, the tip end (the puncture portion 120D) of the infusion needle 120 is inserted to the subcutaneous tissue L3 and indwelling of the needle is stable, the tip end (the puncture portion 120D) is non-hollow and insulin does not flow out to the subcutaneous tissue L3, and thereby, the control of the amount of infusion of insulin into the dermic layer L2 is easier. If the opening of the infusion needle 120 is in the subcutaneous part, the subcutaneous fat may enter the opening and block the opening and the infusion needle 120 may be clogged, however, in the embodiment, the opening (the outflow port 120C) of the infusion needle 120 is in the dermic layer L2, and entrance of the subcutaneous fat into the opening of the infusion needle 120 may be suppressed.

### Movement of Infusion Needle

The infusion needle unit 100 movably supports the infusion needle 120 to prevent, when the infusion needle 120 is indwelled in the living body and the skin expands and contracts, the movement of the tip end within the punctured region of the living body and the action of excessive stress on the punctured region.

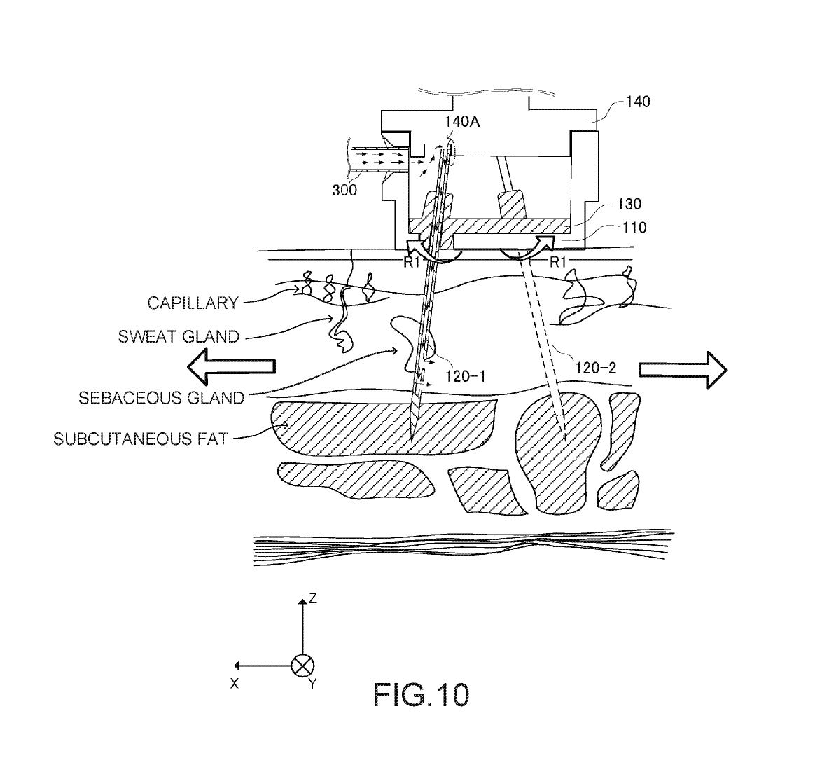

FIG. 10 shows a mode in which the infusion needle 120 moves in a rotation direction R1 (and a horizontal direction) when the skin expands in the horizontal direction by breathing movement or the like. For convenience of explanation, in FIG. 10, the left infusion needle 120 of the plurality of infusion needles 120 is shown by “infusion needle 120-1” and the right infusion needle 120 (shown by dotted lines because the needle is not within the same plane) is shown by “infusion needle 120-2”.

When the skin expands in the horizontal direction, one infusion needle 120-1 and the other infusion needle 120-2 tend to respectively move with the movement of the skin in the punctured region, and the respective tip end sides are subjected to stress by the punctured region in the horizontal directions away from each other. In this regard, if one infusion needle 120-1 and the other infusion needle 120-2 are supported by the supporting member 130 in a fixed position relationship, one infusion needle 120-1 and the other infusion needle 120-2 mutually restrict the movements. Accordingly, one infusion needle 120-1 and the other infusion needle 120-2 act excessive stress against the part to which the punctured region tends to move. Particularly, when the infusion needle 120 is formed using a metal material, the infusion needle 120 does not elastically deform, and thereby, strong stress acts on the skin and pain is caused. Or, in contrast, the infusion needle may bend due to the stress, the flow paths may be blocked, and the chemical supply may be impossible.

On the other hand, the infusion needle 120 according to the embodiment is supported by the supporting member 130 in the position between the tip end and the base end in the longitudinal direction, and movable in the rotation directions, the horizontal directions, and the vertical directions with reference to the initial support position. Further, the plurality of infusion needles 120 are respectively separately supported by the supporting member 130, and the plurality of infusion needles 120 are respectively independently movable in the rotation directions, the horizontal directions, and the vertical directions. That is, when the skin expands in the horizontal direction, the supporting member 130 supporting one infusion needle 120-1 and the other infusion needle 120-2 elastically deforms, and one infusion needle 120-1 and the other infusion needle 120-2 respectively move in the rotation directions and the horizontal directions with movement of the skin as shown in FIG. 10. The infusion needle unit 100 according to the embodiment relaxes the stress (stress in the horizontal direction) acting between the infusion needle 120 and the punctured region and prevents pain and discomfort to the patient by the configuration. In another point of view, bending of the infusion needle 120 may be prevented. Note that the directions in which the infusion needle 120 is movable may be desirably all directions of the rotation directions, the horizontal directions, and the vertical directions, however, may be only one direction in consideration of the expansion and contraction directions of the skin (e.g. the direction nearly perpendicular to the longitudinal direction of the infusion needle 120).

On the other hand, the amount of movement in which the base end of the infusion needle 120 may move is restricted within the predetermined range by the regulating member 140 as a stopper. Specifically, the concave groove portion 140A of the regulating member 140 is provided around the base end of the infusion needle 120, and thereby, when moving in the predetermined range, the base end of the infusion needle 120 comes into contact with the wall surface of the concave groove portion 140A and can no longer move in the direction. The regulating member 140 restricts the movement of the base end of the infusion needle 120 particularly in the rotation directions and prevents the tip end of the infusion needle 120 from moving (tilting) too much in the rotation directions and slipping out by the configuration. In this regard, the stress is dispersed to the respective plurality of infusion needles 120, the plurality of infusion needles 120 respectively move the tip ends in the rotation directions or the horizontal directions in the range in which the needles do not slip out as shown in FIG. 10, and thereby, the range in which the stress acting between the infusion needle 120 and the punctured region (stress in the horizontal direction) may be relaxed is larger. The configuration is particularly effective when the infusion needle 120 is indwelled in the shallow region from the surface of the skin.

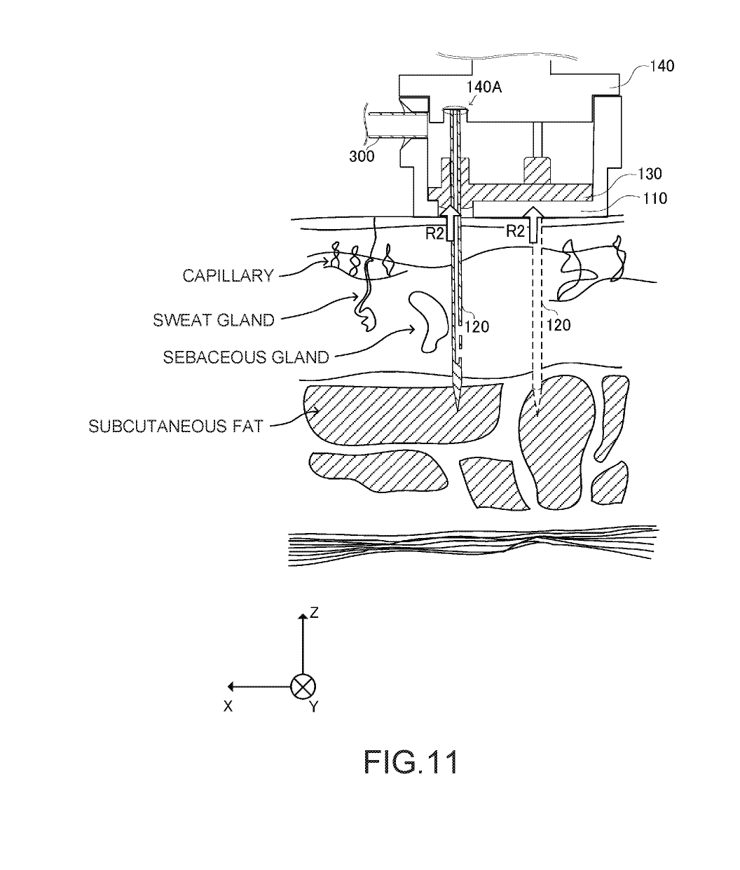

FIG. 11 shows a mode in which the infusion needle 120 moves in a vertical direction R2 when the infusion needle 120 is inserted. The infusion needle 120 is inserted into the living body and indwelled, and the infusion needle 120 having the thinner outer shape (e.g. the outer diameter is 0.18 mm and the inner diameter of the hollow region is 0.11 mm) is used. On the other hand, when the infusion needle 120 is inserted, stress (drag) in the vertical direction acts on the infusion needle 120 from the hard stratum corneum of the epidermis L1, and thereby, if the infusion needle 120 is fixed not via a member for relaxing the stress in the vertical direction, the needle may buckle (bend) due to the stress in the vertical direction.

In this regard, as described above, the infusion needle 120 according to the embodiment has the gap between the base end and the wall surface of the groove portion 140A without stress thereon and supported by the supporting member 130 in the state in which the needle may move in the vertical direction (upwardly). Accordingly, in the infusion needle 120, when the vertical (upward) stress acts from the epidermis L1 at puncture, the supporting member 130 elastically deforms and the stress acting in the vertical direction is dispersed (relaxed). Further, in this regard, after the infusion needle 120 moves in a fixed range (the gap between the base end and the wall surface of the groove portion 140A) in the vertical direction (upwardly), the regulating member 140 serves as a stopper and the needle can be inserted to a desired depth through the epidermis L1. That is, the infusion needle 120 is supported movably in the vertical direction by the supporting member 130 having elasticity, and thereby, buckling is not caused and puncture may be reliably made. Further, by the regulating member 140, indwelling of the infusion needle 120 in the shallow region of the epidermis L1 and upward movement and slipping out due to the stress from the punctured region may be prevented.

As described above, according to the infusion needle unit 100 of the embodiment, the position of the infusion needle 120 between the tip end and the base end in the longitudinal direction is supported by the supporting member 130 having elasticity so that the infusion needle 120 may move in the horizontal direction, and thereby, the stress between the infusion needle 120 and the punctured region (stress in the horizontal direction) generated due to expansion and contraction of the skin may be relaxed and pain and discomfort to the patient may be prevented. Further, the infusion needle 120 is supported by the supporting member 130 with the gap between the base end and the wall surface of the groove portion 140A to be movable in the vertical direction (upwardly), and the stress in the vertical direction generated in the infusion needle 120 at puncture may be relaxed and buckling of the infusion needle 120 may be prevented.

In addition, according to the infusion needle unit 100 of the embodiment, the amount of movement of the infusion needle 120 may be regulated (restricted) in the predetermined range by the regulating member 140, and thereby, slipping out during indwelling may be prevented.

### Second Embodiment

An infusion needle unit 100′ according to the embodiment is different from that of the first embodiment in a position relationship and a contact structure between a base end of a infusion needle 120′ and a concave groove portion 140A′ of a regulating member 140′. In the embodiment, the configurations except the infusion needle 120′ and the regulating member 140′ are common with the liquid infusion apparatus according to the first embodiment, and the explanation of the configurations will be omitted.

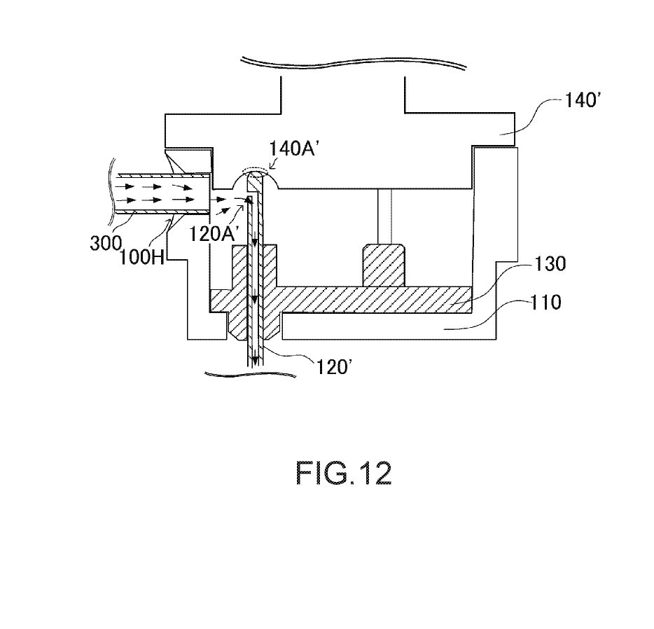

FIG. 12 shows the position relationship between the infusion needle 120′ and the regulating member 140′ in the contact part of the infusion needle unit 100′ according to the embodiment. In the embodiment, in order to stabilize the position of the infusion needle 120′ using a friction force, the base end of the infusion needle 120′ and the concave groove portion 140A′ of the regulating member 140′ are constantly in contact as shown in FIG. 12.

More specifically, the base end of the infusion needle 120′ has a curved shape convex outward, and the needle is supported by the supporting member 130 so that the curved shape of the base end of the infusion needle 120′ may be in contact with the concave groove portion 140A′ of the regulating member 140′. The concave groove portion 140A′ of the regulating member 140′ takes on a curved shape concave inward. The curved shape of the concave groove portion 140A′ and the curved shape of the base end of the infusion needle 120′ are placed to face each other and both are in contact in the states including the state without stress acting on the infusion needle 120′. That is, the infusion needle 120′ moves with contact between the base end of the infusion needle 120′ and the concave groove portion 140A′. The structure with contact between the curved shape convex outward and the curved shape concave inward is for guiding the infusion needle 120′ to smoothly move in a predetermined rotation direction and allowing a constant friction force to act at the movement. In other words, the regulating member 140′ according to the embodiment regulates the amounts of movement of the base end of the infusion needle 120′ in the vertical directions and the horizontal directions within a predetermined range so that a constant friction force may act.

When a force for holding the position of the infusion needle 120 does not act, the infusion needle 120 is unstably positioned and easy to move. As a result, the infusion needle 120 may frequently move and may cause pain and discomfort to the patient. Accordingly, in the embodiment, the base end of the infusion needle 120′ and the concave groove portion 140A′ are brought into contact and a constant friction force is allowed to act on the movement of the base end of the infusion needle 120′. In other words, the infusion needle 120′ may move only when a force more than the friction force acts on the infusion needle 120′, and thereby, the position of the infusion needle 120 is stably held.

Note that, in the case of the infusion needle 120′ according to the embodiment, the base end of the infusion needle 120′ is constantly in contact with the wall surface of the groove portion 140A′, and thereby, if the inflow port 120A is formed in the base end, the inflow port 120A may be blocked by the wall surface of the groove portion 140A′ and the liquid may not flow therein. Accordingly, it is desirable that an inflow port 120A′ is provided in a region between the position supported by the supporting member 130 and the base end of the infusion needle 120′ to communicate with a needle part flow path 120B′ as shown in FIG. 12.

In the embodiment, like the first embodiment, it is desirable to form the concave groove portion 140A′ to restrict the range in which the base end of the infusion needle 120′ is movable in the rotation directions in order to prevent the tip end of the infusion needle 120′ from moving (tilting) too much in the rotation directions and slipping out. Further, like the first embodiment, in order to relax the stress acting in the vertical direction at puncture, a gap may be provided between the base end of the infusion needle 120′ and the concave groove portion 140A′ of the regulating member 140′ in an initial support position. In this case, the base end of the infusion needle 120′ may come into contact with the concave groove portion 140A′ of the regulating member 140′ by the repulsive force acting from the punctured region in the vertical direction when the infusion needle 120′ is indwelling.

As described above, according to the infusion needle unit 100′ of the embodiment, the base end of the infusion needle 120′ having the curved shape convex outward moves in contact with the regulating member 140′ having the curved shape concave inward, and thereby, stability of the position of the infusion needle 120′ may be improved.

### Other Embodiments

In the above described respective embodiments, the configuration of infusing insulin into the living body is shown. However, the liquid infusion apparatus according to the invention may be used for various chemicals. For example, glucagon, growth hormone etc. are preferable as chemicals continuously infused into the dermic layer, and morphine etc. are preferable as chemicals continuously infused into the subcutaneous tissue.

Further, in the above described respective embodiments, the configuration of providing the plurality of infusion needles 120 is shown. However, the same advantages as those of the embodiments may be expected even when the liquid infusion apparatus according to the invention has a single infusion needle 120. This is because, even in the case of a single infusion needle 120, the infusion needle 120 may act excessive stress on the punctured region and move the needle tip within the punctured region depending on the relative position relationship with the adhesive pad 1A fixed to the abdomen of the human body or the like, and pain and discomfort may be caused to the patient.

Furthermore, in the above described respective embodiments, the configuration of inserting the infusion needle 120 to penetrate the elastic member of rubber or the like from above to below and bonding the elastic member and the infusion needle 120 is shown as the configuration of supporting the infusion needle 120 by the supporting member 130. However, other configurations may be employed as long as the infusion needle 120 may move at least in the directions (horizontal directions) in which the skin expands and contracts. For example, the configuration may be formed by a cylindrical fixing member fitted on the outer circumferential surface of the infusion needle 120 and an elastic member of rubber or the like for fixing the fixing member to the liquid holding member, and the infusion needle 120 may be movably supported by fitting of the infusion needle 120 in the fixing member. In addition, the infusion needle 120 may be attached to the elastic member by a technique of welding the elastic member and the infusion needle 120 or a technique of press fitting of the infusion needle 120 in the elastic member and supported. Or, the supporting member 130 may be formed using a spring having elasticity in place of the rubber member.

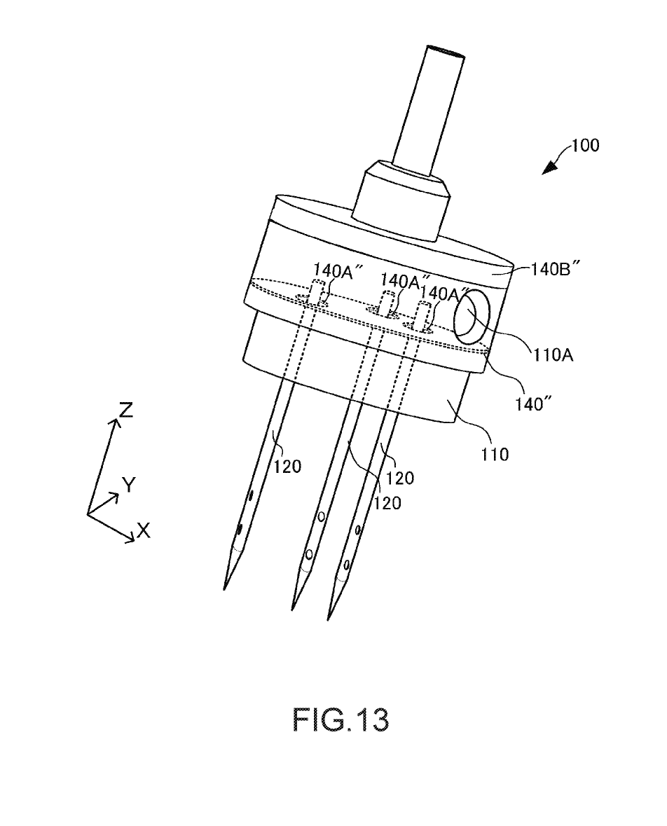

In the above described respective embodiments, as the configuration of the regulating member 140, the configuration in which the concave groove portion 140A formed in the lid-like member regulates the amount of movement of the base end of the infusion needle 120 within the predetermined range is shown. However, the regulating member 140 may have another configuration as long as the member may regulate the movement range of the base end of the infusion needle 120. For example, as shown in FIG. 13 (by dotted lines), a regulating member 140″ may be a member that regulates the movement range of the base end of the infusion needle 120 by surrounding the base end side of the infusion needle 120 in a ring form. Or, as shown in FIG. 13, a lid member 140B″ covering the housing space 110B and the regulating member 140″ may be formed as separate members. On the other hand, when slipping out of the infusion needle 120 is not considered as a problem, the regulating member 140 is not necessarily provided.

Further, in the above described respective embodiments, as the configuration of the infusion needle 120, the configuration in which the puncture portion 120D is provided in the tip end and the outflow port 120C laterally penetrates the side wall of the member at the tip end side of the infusion needle 120 is shown. However, the infusion needle 120 may have no puncture portion 120D as long as a certain degree of stability is expected in the indwelling state. In this case, the outflow port 120C may penetrate the tip end of the infusion needle 120 in the longitudinal direction. In addition, in place of the configuration in which the infusion needle 120 is inserted in the perpendicular direction to the surface of the living body, the infusion needle 120 may be attached to extend in the oblique direction with respect to the adhesive surface of the adhesive pad 1A (e.g. at an angle tilted by 30 degrees from the downward direction) and diagonally inserted into the surface of the living body and indwelled.

Furthermore, in the above described respective embodiments, as the configuration of the fixing member for fixing the liquid infusion apparatus to the living body, the configuration using the adhesive pad 1A is shown. However, the fixing member may be another than the adhesive pad 1A, e.g. a fixing member using a hook-and-loop fastener to be wrapped around an arm or the like.

In the above described respective embodiments, as the configuration of the liquid infusion apparatus, the configuration in which the infusion needle unit 100 is housed within the casing 1 and integrated with the liquid transport part 400 etc. is shown. However, the infusion needle unit 100 may be attached to the casing 1 with the supply tube 300 inserted into the liquid holding member 110 of the infusion needle unit 100 in use.

As above, the specific examples of the invention are explained in detail, however, they are just examples and do not limit the scope of the appended claims. The technology described in the appended claims includes various modifications and alternations of the exemplified specific examples.

The entire disclosure of Japanese Patent Application No. 2015-082285 filed Apr. 14, 2015 is expressly incorporated by reference herein.

What is claimed is:

1. An infusion needle unit comprising:
an infusion needle in which a flow path of a liquid is provided;
a supporting member having elasticity and supporting a position between a tip end and a base end of the infusion needle in a longitudinal direction; and
a regulating member that regulates a movement of the base end of the infusion needle.

2. The infusion needle unit according to claim 1, further comprising a liquid holding part that holds the liquid while supporting the supporting member and the regulating member,
wherein the infusion needle is provided with an inflow port communicating with the flow path in the base end, and
the liquid held in the liquid holding part flows into the infusion needle from a gap between the base end of the infusion needle and the regulating member via the inflow port.

3. The infusion needle unit according to claim 1, wherein the regulating member regulates a movement of the infusion needle from the tip end toward the base end.

4. The infusion needle unit according to claim 1, wherein the supporting member supports the infusion needle so that the base end of the infusion needle may move in contact with the regulating member,
the base end of the infusion needle takes on a curved shape convex outward, and
a surface of the regulating member in contact with the base end of the infusion needle takes on a curved shape concave inward.

5. The infusion needle unit according to claim 4, wherein the infusion needle is provided with an inflow port that allows the liquid to flow into the flow path between the position supported by the supporting member and the base end.

6. The infusion needle unit according to claim 1, the supporting member supports a plurality of the infusion needles.

7. A liquid infusion apparatus comprising:
an infusion needle in which a flow path of a liquid is provided;
a supporting member having elasticity and supporting a position between a tip end and a base end of the infusion needle in a longitudinal direction;
a regulating member that regulates a movement of the base end of the infusion needle; and
a pump that transports the liquid.

8. The liquid infusion apparatus according to claim 7, further comprising a liquid holding part that holds the liquid while supporting the supporting member and the regulating member,
wherein the infusion needle is provided with an inflow port communicating with the flow path in the base end, and
the liquid held in the liquid holding part flows into the infusion needle from a gap between the base end of the infusion needle and the regulating member via the inflow port.

9. The liquid infusion apparatus according to claim 7, wherein the regulating member regulates a movement of the infusion needle from the tip end toward the base end.

10. The liquid infusion apparatus according to claim 7, wherein the supporting member supports the infusion needle so that the base end of the infusion needle may move in contact with the regulating member,
the base end of the infusion needle takes on a curved shape convex outward, and
a surface of the regulating member in contact with the base end of the infusion needle takes on a curved shape concave inward.

11. The liquid infusion apparatus according to claim 7, wherein the infusion needle is provided with an inflow port that allows the liquid to flow into the flow path between the position supported by the supporting member and the base end.

12. The liquid infusion apparatus according to claim 7, wherein the supporting member supports a plurality of the infusion needles.
